(19) United States
(12) Patent Application Publication
(10) Pub. No.: US 2003/0191382 A1
(43) Pub. Date: Oct. 9, 2003

(54) Non-contact tonometer

(76) Inventors: David A. Luce, Clarence Center, NY (US); Bruce Siskowski, Orchard Park, NY (US)

Correspondence Address: George L. Snyder, Jr., Hodgson Russ LLP, Suite 2000, One M&T Plaza, Buffalo, NY, 14203-2391, US

(21) Appl. No.: 10/117,750

(22) Filed: Apr. 5, 2002

(51) Int. Cl. A61B 3/16

(52) U.S. Cl. 600/401

(57) ABSTRACT

A non contact tonometer of a type having a fluid pump in flow communication with a fluid discharge tube for directing a fluid pulse at an eye of a patient to observably transfigure the cornea is improved by providing the fluid discharge tube with a flared inlet portion for reducing inlet losses. In a preferred embodiment, the flared inlet portion is defined by a circumferential internal radius.

### FIELD OF THE INVENTION

The present invention relates generally to ophthalmic instruments, and more particularly to non-contact tonometers that measure intraocular pressure (IOP) by directing a fluid pulse at an eye to transfigure the cornea.

### BACKGROUND OF THE INVENTION

Non-contact tonometers are well-known in the field of ophthalmology for measuring intraocular pressure (IOP) by directing a fluid pulse at the cornea to cause observable deformation of the cornea. Most commonly, the observable deformation is a flattening of a predetermined area of the cornea, a condition known as applanation. In prior art non-contact tonometers, the fluid pulse is generated by a fluid pump mechanism defining a plenum chamber for pressurized fluid. In order to direct the fluid pulse at the patient's cornea, a narrow cylindrical fluid discharge tube (also commonly referred to in the art as a “nozzle”) has an inlet orifice in flow communication with the plenum chamber and an outlet orifice that is aligned relative to the eye during testing.

Since the early 1960s, when non-contact tonometers underwent initial development, through the present day, in which non-contact tonometers are very widely used by ophthalmic practitioners as a fundamental diagnostic tool, the shape of the fluid discharge tube has remained the same. Specifically, a length of narrow cylindrical tubing having flat ends and an axial flow passageway has been used faithfully as a de facto standard in non-contact tonometers to direct a fluid pulse from the plenum chamber to the eye. For example, U.S. Pat. No. 3,585,849 issued Jun. 22, 1971 gives an illustration of this universally adopted style of discharge tube at FIGS. 1 and 2, and further examples of this basic configuration can be readily found throughout the patent literature. While this type of discharge tube is inexpensive to manufacture and delivers a well-directed fluid pulse, it is not optimal from the standpoint of efficient fluid dynamics because of “vena cava” inlet losses and accompanying flow instability.

An exception to the aforementioned de facto standard can be found in U.S. Pat. No. 3,181,351 to Stauffer. FIG. 2 of this patent shows a non-contact tonometer that includes an end cap 144 having a plurality of grooves 146 extending along an internal conic surface thereof. The end cap 144 is threadably connected to an end member 140 having a frusto-conical projection 142, such that the outer surface of frusto-conical projection 142 cooperates with the grooved conic surface of the end cap to provided a plurality of convergent fluid nozzles aimed at a common point. A fluid pump (air puff generator 152) produces pressurized air within a plenum volume defined by an annular groove 148 in end cap 144 and the exterior of end member 140, such that airflows through grooves 146 toward a common target point. The arrangement taught by U.S. Pat. No. 3,181,351 is complex to manufacture, and it situates the convergent flow grooves in space that in present-day instruments is occupied by emitters and detectors used for monitoring corneal transfiguration and aligning the instrument relative to an eye.

Finally, U.S. Pat. No. 4,386,611 entitled “Tonometer With Improved Fluid Discharge Tube” teaches positioning the fluid discharge tube such that almost 40% of the total length of the tube extends into the plenum chamber to disrupt wavefronts, and texturizing the inner wall of the tube to enhance air pulse consistency. This does not, however, teach or suggest reshaping the fluid discharge tube. In fact, the patent states at column 2, lines 9-12, that “[v]arious modifications to the discharge tube such as tapering the end of the internal wall of the tube toward the exterior well in the vicinity of the plenum chamber have also been tried with very limited success.” Thus, this patent leads persons skilled in the art away from modifying the shape of the tube to achieve a meaningful improvement in performance.

### SUMMARY OF THE INVENTION

Therefore, it is an object of the present invention to provide a non-contact tonometer with an improved fluid discharge tube that decreases inlet losses to provide a more stable and repeatable fluid pulse and reduce overall pump requirements.

It is another object of the present invention to provide a non-contact tonometer with an improved fluid discharge tube that is well-suited for use in a lightweight, handheld instrument.

It is another object of the present invention to achieve the aforementioned objects without a substantial increase in manufacturing complexity and cost for the improved fluid discharge tube.

A non-contact tonometer formed in accordance with a preferred embodiment of the present invention comprises an improved fluid discharge tube for alignment relative to an eye of a patient. The fluid discharge tube has a flared inlet portion and an outlet orifice spaced from the inlet portion along a test axis defined by the discharge tube. The tonometer further comprises a fluid pump system having a plenum chamber communicating with the flared inlet portion of the fluid discharge tube, whereby a fluid pulse is generated and directed along the test axis to transfigure the cornea. The tonometer is equipped with applanation detection means for monitoring deformation of the cornea and providing applanation signal information indicative of a state of applanation of the cornea caused by the fluid pulse, and with means for determining a fluid pressure within the plenum chamber corresponding to applanation of the cornea. Processing means correlates the plenum fluid pressure with an intraocular pressure of the eye.

In the preferred embodiment, the flared inlet portion of the discharge tube is defined by an internal circumferential radius. In an alternative embodiment, the flared inlet portion is characterized by a frusto-conical internal configuration. The discharge tube of either embodiment has a uniform wall thickness along its entire length and can be formed of a unitary piece of tubing stock.

The improvement of the present invention lowers the necessary plenum pressure for achieving applanation, thereby reducing fluid pump demands. Consequently, a lighter pump system may be employed that produces less “overpuff” on the eye and is ergonomically beneficial for a handheld instrument. Moreover, instrument performance is improved by a more stable fluid pulse.

### BRIEF DESCRIPTION OF THE DRAWINGS

The nature and mode of operation of the present invention will now be more fully described in the following detailed description of the invention taken with the accompanying drawing figures, in which:

FIG. 1 is a perspective view of a non-contact tonometer formed in accordance with a preferred embodiment of the present invention;

FIG. 2 is a schematic diagram of the non-contact tonometer shown in FIG. 1;

FIG. 3 is a cross-sectional view of a nosepiece and associated fluid pump of the non-contact tonometer shown in FIG. 1;

FIG. 4 is an enlarged cross-sectional view of a fluid discharge tube formed in accordance with a preferred embodiment of the present invention; and

FIG. 5 is an enlarged cross-sectional view of a fluid discharge tube formed in accordance with another embodiment of the present invention.

### DETAILED DESCRIPTION OF THE INVENTION

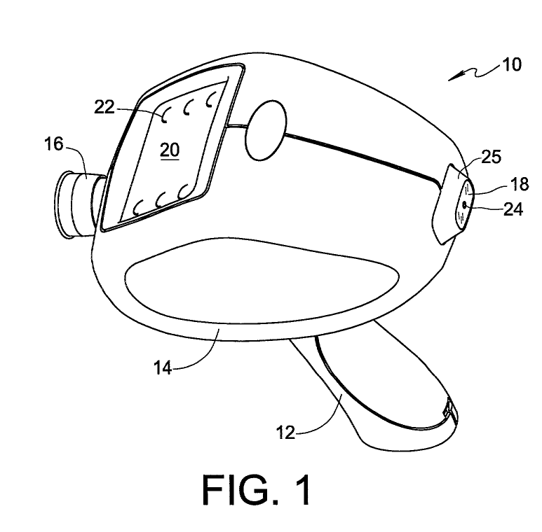

FIG. 1 of the drawings shows a non-contact tonometer (NCT) 10 embodying the present invention. NCT 10 is depicted as being a handheld instrument having a handle portion 12 and a head portion 14 at the top of the handle portion. While the present invention is described in the context of a handheld NCT, it can also be embodied in a table-top NCT. Handle portion 12 houses a rechargeable power source for energizing alignment and tonometric measurement systems carried by head portion 14. Also visible in FIG. 1 is an operator eyepiece 16 at one end of head portion 14, a front window 18 at an opposite end of head portion 14 for facing a patient, and a liquid crystal display 20 with pushbutton control overlay 22 angled toward the operator near operator eyepiece 16.

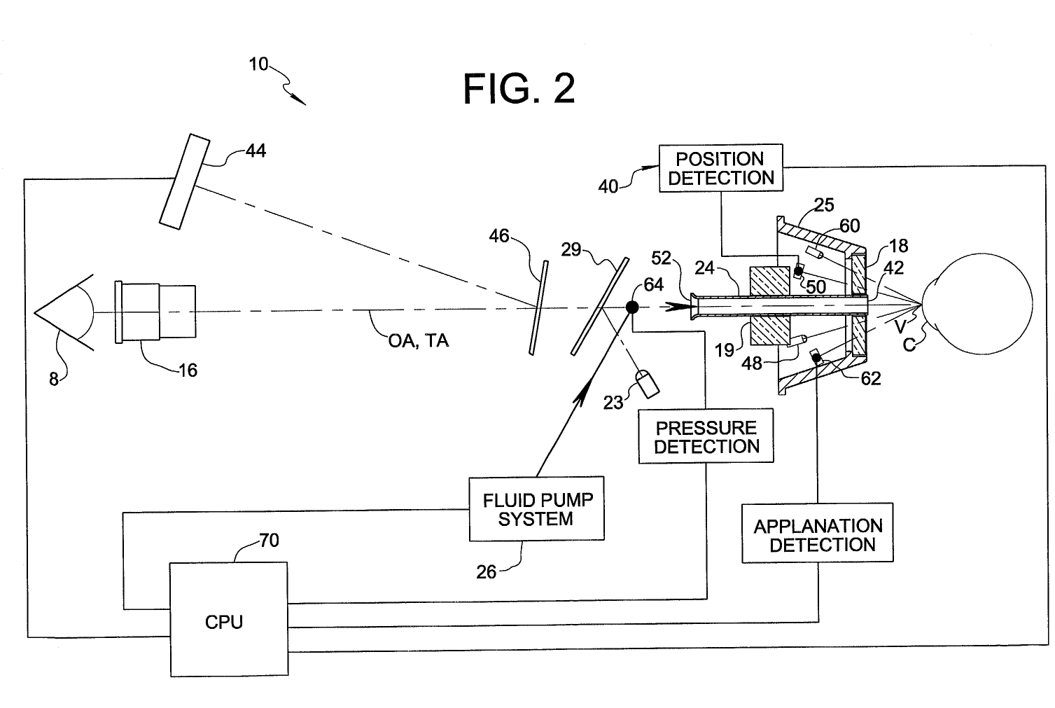

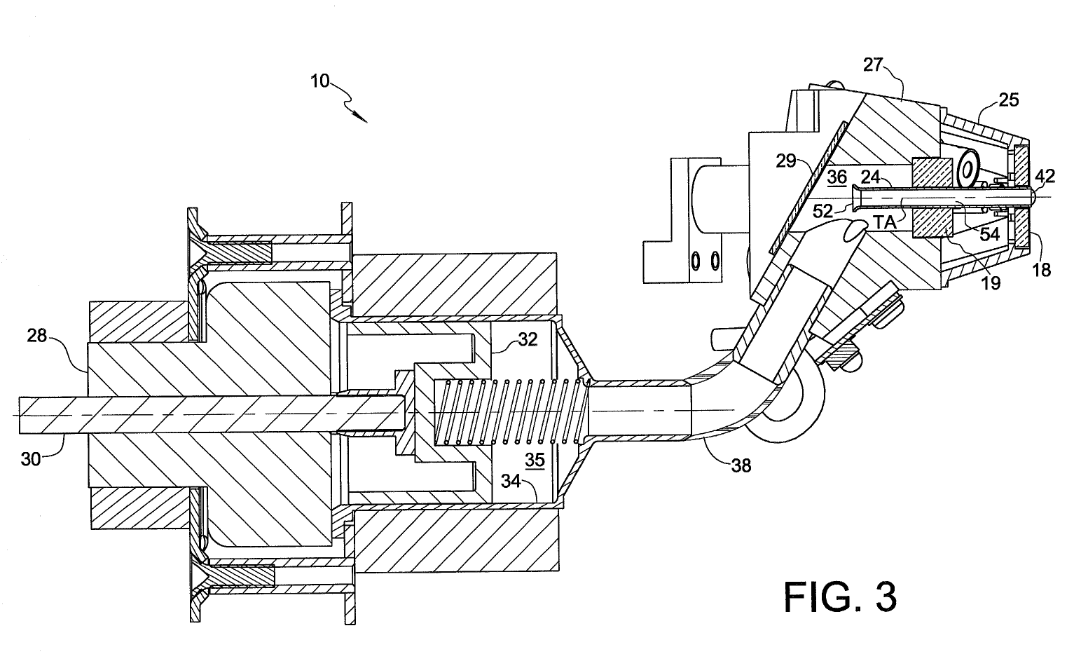

FIG. 2 provides a schematic representation of the alignment and tonometric measurement systems housed by head portion 14. Non-contact tonometer 10 is operable to discharge a fluid pulse through a fluid discharge tube 24 aligned along a test axis TA to cause observable deformation of a patient's cornea C for purposes of measuring intraocular pressure. The fluid pulse is generated by a fluid pump system 26 communicating with fluid discharge tube 24, which extends through a nosepiece 25 fixed to a mounting member 27 seen in FIG. 3. A currently preferred fluid pump system is shown in FIG. 3 and comprises a linear solenoid 28 having a plunger 30, a piston 32 driven by plunger 30 and slidably received by a corresponding cylinder 34 to compress air within a compression chamber 35 when solenoid 28 is energized, and a plenum chamber 36 in flow communication with compression chamber 35 by way of a fluid conduit 38. Fluid discharge tube 24 extends into and communicates with plenum chamber 36.

Of course, those familiar with the art of non-contact tonometers will realize that other fluid pump systems are also possible. By way of example, a pump system comprising a rotary solenoid connected to a piston by a pivotal linkage may be employed to compress fluid within a plenum chamber of the pump system with which the fluid discharge tube communicates. Other systems are also possible, including systems wherein a piston is driven by force supplied by a mechanical spring. It will be understood that the term “fluid pump” is not limited to the preferred fluid pump system described herein, and includes any system that functions to compress fluid.

As a prerequisite to testing, it is necessary for an operator 8 to align NCT 10 in three dimensions (X-Y-Z alignment) relative to the patient's eye. The patient is instructed to gaze at a target image presented along optical axis OA by a target light source 23 and a beam splitter 29. The operator 8 is preferably guided in coarse alignment of NCT 10 by viewing the patient's eye through operator eyepiece 16 along an optical axis OA that coincides with test axis TA. A planar-planar objective lens 19 on optical axis OA cooperates with front window 18 to support fluid discharge tube 24 without blocking the operator's view of the patient's eye. In a preferred embodiment, an opto-electronic position detection system 40 associated with nosepiece 25 senses the position of an outlet orifice 42 of fluid discharge tube 24 relative to a corneal vertex V and provides signal information used to drive an instructive “heads up” display 44 providing real time X, Y, and Z alignment cues. An image of instructive display 44 is projected to the operator along optical axis OA by a beam splitter 46, such that the instructive display image is optically superimposed with an image of the patient's eye as viewed by the operator. Proper alignment is confirmed by position detection system 40. Reference numerals 48 and 50 respectively denote an emitter and a detector of position detection system 40. Commonly owned U.S. patent application Ser. No. 09/992,875, filed Nov. 6, 2001 and incorporated herein by reference in its entirety, describes a preferred alignment system in greater detail at paragraphs [0022] through [0036] and FIGS. 3-10.

Alternative means for aligning NCT 10 are also possible. By way of non-limiting example, NCT 10 may include an alignment system as taught in U.S. Pat. No. 4,881,807, wherein the operator views a video display of the eye with superimposed instructional graphics. If NCT 10 is designed as an inexpensive screening tool wherein measurement accuracy requirements can be relaxed to reduce cost, it is conceivable to have a “go/no go” alignment system that simply confirms proper alignment without providing any instructional display or graphics to the operator. An example of a “go/no go” alignment system is described in commonly owned U.S. Pat. No. 6,361,495.

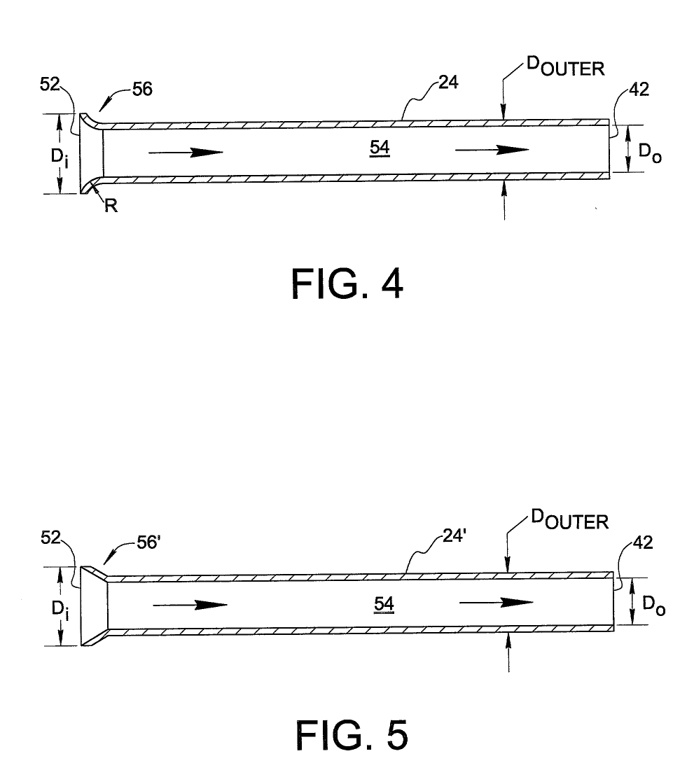

Once proper alignment of NCT 10 is achieved, fluid pump system 26 is triggered to generate a fluid pulse. Referring to FIG. 3, it will be seen that plenum chamber 36 is provided by an axial hole through mounting member 27 and further defined by beam splitter 29 and objective lens 19. In addition, it will be seen that fluid discharge tube 24 comprises an inlet orifice 52 and an axially extending fluid passageway 54 connecting inlet orifice 52 with outlet orifice 42. In accordance with the present invention, and as best seen in the enlarged view of FIG. 4, fluid discharge tube 24 includes a flared inlet portion 56 beginning at inlet orifice 52. The inner diameter of fluid discharge tube 24, which begins as Dᵢ at inlet orifice 52, decreases continuously through inlet portion 56 in a direction of flow toward outlet orifice 42. In the embodiment illustrated in FIG. 4, the inner diameter decreases as a nonlinear function of the axial distance along the tube until it reaches a final diameter Dₒ associated with the remainder of the tube length and outlet orifice 42. A nonlinear reduction in the inner diameter allows a smooth transition to the final outlet diameter Dₒ that avoids discontinuities or steps in the inner wall surface of discharge tube 24, which is a desirable condition with respect to the fluid dynamic properties of the system. Flared inlet portion 56 in FIG. 4 is preferably defined by a circumferential internal radius R, which enables fluid discharge tube 24 to be manufactured in a simple manner. In a preferred method for manufacturing fluid discharge tube 24, a piece of stainless steel stock tubing is secured in a lathe or drill chuck and rotated. While the tube is spinning, it is forced in an axial direction against an axially aligned spherical ball to form the flared inlet portion and circumferential internal radius seen in FIG. 4. The radially expanded outer edge about inlet orifice is then finish turned or otherwise machined to produce a desired diameter that will not substantially interfere with the tonometer operator's view of the eye. The piece of stock tubing is then cut to length.

The table below lists the dimensions of a currently preferred fluid discharge tube manufactured to embody the present invention:

| Length (L) | 1.064 inches | | |
|---|---|---|---|
| Outer Diameter (D_{OUTER}) | 0.120 inches | | |
| Inner Diameter at Inlet (Dᵢ) | 0.160 inches | | |
| Inner Diameter at Outlet (Dₒ) | | +.0006 | |
| | 0.0950 | | inches |
| | | −.0000 | |

| | |
|---|---|
| Flare Radius (R) | 0.050 inches |

Prototype testing of non-contact tonometers outfitted with a flared discharge tube having the above dimensions has demonstrated significant reduction in the plenum pressure required to achieve applanation of an eye at a given IOP, as compared with the plenum pressure required to applanate the same eye in a tonometer using a straight fluid discharge tube of the prior art. This reduction is attributed to a decrease in inlet losses as fluid enters the discharge tube from the plenum chamber through the flared inlet portion. As a result of the lower plenum pressure requirements associated with the present invention, smaller and lighter fluid pump components can be used, i.e. a smaller solenoid or other electro-motive driver and a lighter piston may be used. It is known that piston momentum contributes to an uncomfortable “overpuff” in excess of that needed to cause applanation, and that decreasing the kinetic energy of the system will reduce this overpuff. Thus, the present invention provides a comfort benefit to patients. Moreover, because much of the overall weight of a non-contact tonometer is attributed to the components of the fluid pump system, the present invention helps to make a lightweight handheld instrument a practical reality. A further benefit of the present invention is that a more consistent and stable fluid pulse is generated owing to reduced inlet losses, thereby improving the ability of the instrument to give reproducible measurements.

FIG. 5 depicts a fluid discharge tube 24′ formed in accordance with an alternative embodiment of the present invention. Discharge tube 24′ differs from discharge tube 24 of the first embodiment in that the inner diameter decreases linearly through inlet portion 56′ in a direction of flow, thereby forming a frusto-conical inlet portion.

The configurations of inlet portion 56 in FIG. 4 and inlet portion 56′ in FIG. 5 do not involve tapering the end of the internal wall of the tube toward the exterior in the vicinity of the plenum chamber, as mentioned in U.S. Pat. No. 4,386,611 cited in the Background of the Invention section herein. Tapering the internal wall keeps a constant outer diameter, however it requires a reduction in the wall thickness in an already thin tubing wall, and thus offers minimal opportunity for improvement of flow conditions. In discharge tubes 24 and 24′ of the present invention, the thickness of the tube wall remains constant along the entire length of the discharge tube, including the flared inlet portion.

A preferred arrangement for optically detecting applanation of cornea C is shown schematically in FIG. 2. An infra-red emitter 60 is mounted in nosepiece 25 and obliquely aimed at corneal vertex V, and a photosensitive detector 62 is located on the opposite side of optical axis OA facing corneal vertex V along an oblique direction symmetrically opposite to that of applanation emitter 60. A collector lens (not shown) and a pinhole diaphragm (also not shown) are positioned in front of applanation detector 62, which is located in the focal plane of the collector lens. When the cornea C is in its normal convex shape, parallel incident rays from emitter 60 are reflected in a fanned-out fashion by the curved corneal surface, and a weak detection signal is generated at applanation detector 62. As a portion of the corneal surface approximates a flat surface at applanation, the incident parallel beam is reflected by the flat surface as a parallel beam in the direction of the collector lens, which focuses the beam through the pinhole diaphragm and onto the surface of applanation detector 62. As a result, applanation detector 62 registers a peak detection signal corresponding to applanation. Those familiar with non-contact tonometers will recognize that this arrangement for optically detecting applanation is already known from the prior art.

Tonometric measurement involves correlation of the pressure within plenum chamber 36 at applanation with IOP. Therefore, a pressure sensor 64, for example a pressure transducer or the like, is located within plenum chamber 36 for generating signal information indicative of the fluid pressure within the plenum chamber. As an alternative to directly measuring plenum pressure using a pressure sensor, it is possible to indirectly measure plenum pressure by driving the fluid pump system 26 such that the pressure within plenum chamber 36 increases as a known function of time, and measuring the time required to achieve applanation as a correlate to IOP.

The analog signal information from pressure sensor 64 and applanation detector 62 is filtered and converted to digital form for processing by a central processing unit (CPU) 70. The plenum pressure at the time of applanation is then correlated to IOP by CPU 70. IOP measurement data are reported to the operator by liquid crystal display 20, and can be transmitted, preferably by wireless transmission, to a printing device and/or a remote computer.

What is claimed is:

1. In a non-contact tonometer of a type having a fluid pump in flow communication with a fluid discharge tube for directing a fluid pulse at an eye of a patient to transfigure a cornea of said eye, the improvement comprising:
said fluid discharge tube having a flared inlet portion.

2. The improvement according to claim 1, wherein said fluid discharge tube has an inner diameter that decreases continuously and non-linearly through said inlet portion in a direction of flow.

3. The improvement according to claim 2, wherein said flared inlet portion is defined by a circumferential internal radius.

4. The improvement according to claim 1, wherein said fluid discharge tube has a uniform wall thickness along its entire length.

5. The improvement according to claim 1, wherein said fluid discharge tube is formed from a unitary piece of cylindrical tubing.

6. In a non-contact tonometer of a type having a fluid pump in flow communication with a fluid discharge tube for directing a fluid pulse at an eye of a patient to transfigure a cornea of said eye, said fluid discharge tube having an inlet orifice in flow communication with said fluid pump, an outlet orifice for alignment relative to said eye, and a fluid passageway connecting said inlet orifice and said outlet orifice, the improvement comprising:
said inlet orifice being a circular orifice having a diameter Dᵢ of approximately 0.160 inches, and said outlet orifice being a circular orifice having a diameter Dₒ of approximately 0.0953 inches.

7. The improvement according to claim 6, wherein said fluid discharge tube is at least one inch in length from said inlet orifice to said outlet orifice.

8. The improvement according to claim 6, wherein said fluid discharge tube is formed from a unitary piece of cylindrical tubing.

9. A non-contact tonometer comprising:
a fluid pump system;
a fluid discharge tube having a flow axis for alignment relative to an eye of a patient, a flared inlet portion in flow communication with said fluid pump system, and an outlet orifice spaced from said inlet portion along said flow axis;
a light source spaced from said fluid discharge tube for emitting a beam of light toward said eye for reflection by a cornea of said eye;
a light sensitive detector arranged to receive corneally reflected light and provide applanation signal information indicative of corneal transfiguration caused by a fluid pulse generated by said fluid pump and directed at said eye through said fluid discharge tube;
a pressure transducer arranged to detect fluid pressure within said plenum chamber and provide pressure signal information indicative of said plenum pressure; and
signal processing means for receiving said applanation signal information and said pressure signal information and calculating an intraocular pressure value therefrom.

10. The non-contact tonometer according to claim 9, wherein said fluid discharge tube has an inner diameter that decreases continuously and non-linearly through said inlet portion in a direction of flow.

11 The non-contact tonometer according to claim 10, wherein said flared inlet portion is defined by a circumferential internal radius.

12. The non-contact tonometer according to claim 9, wherein said fluid discharge tube has a uniform wall thickness along its entire length.

13. The non-contact tonometer according to claim 9, wherein said fluid discharge tube is formed from a unitary piece of cylindrical tubing.

14. The non-contact tonometer according to claim 9, wherein said non-contact tonometer is a handheld non-contact tonometer.

15 A non-contact tonometer comprising:
a fluid discharge tube having a test axis for alignment relative to an eye of a patient, a flared inlet portion, and an outlet orifice spaced from said inlet portion along said test axis;
fluid pump means having a plenum chamber communicating with said flared inlet portion of said fluid discharge tube, said fluid pump means generating a fluid pulse for direction by said fluid discharge tube along said test axis to transfigure a cornea of said eye;
applanation detection means for monitoring said cornea and providing applanation signal information indicative of a state of applanation of said cornea caused by said fluid pulse;
means for determining a fluid pressure within said plenum chamber corresponding to said state of applanation of said cornea; and
means for correlating said fluid pressure with an intraocular pressure of said eye.

16. The non-contact tonometer according to claim 15, wherein said fluid discharge tube has an inner diameter that decreases continuously and non-linearly through said inlet portion in a direction of flow.

17 The non-contact tonometer according to claim 16, wherein said flared inlet portion is defined by a circumferential internal radius.

18. The non-contact tonometer according to claim 15, wherein said fluid discharge tube has a uniform wall thickness along its entire length.

19. The non-contact tonometer according to claim 15, wherein said fluid discharge tube is formed from a unitary piece of cylindrical tubing.

20. The non-contact tonometer according to claim 15, wherein said non-contact tonometer is a handheld non-contact tonometer.
